(19) United States
(12) Patent Application Publication
(10) Pub. No.: US 2005/0112113 A1
(43) Pub. Date: May 26, 2005

(54) Presbyopia treatment by lens alteration

(76) Inventors: Jonathan S. Till, Roanoke, VA (US); Ronald D. Blum, Roanoke, VA (US)

Correspondence Address: KENYON & KENYON, 1500 K STREET, N.W., SUITE 700, WASHINGTON, DC, 20005, US

(21) Appl. No.: 10/969,868

(22) Filed: Oct. 22, 2004

Related U.S. Application Data
(63) Continuation-in-part of application No. 10/050,879, filed on Jan. 18, 2002.
Application No. 10/050,879 is a continuation-in-part of application No. 09/930,287, filed on Aug. 16, 2001, now abandoned.
(60) Provisional application No. 60/262,423, filed on Jan. 19, 2001.
(60) Provisional application No. 60/225,659, filed on Aug. 16, 2000.

(51) Int. Cl. A61K 38/43; A61F 2/14

(52) U.S. Cl. 424/94.1; 424/427

(57) ABSTRACT

Embodiments of the present invention relate to methods and pharmacological compositions to treat presbyopia in the human eye. According to the embodiments, pharmacological compositions may be applied to an eye to effect a change in the accommodative ability of the eye by the breaking and reduction of lenticular bonds in the eye that may be responsible for presbyopia. The compositions may be applied in an inactive state and subsequently be activated to achieve a therapeutic effect.

This application is a continuation-in-part of U.S. application Ser. No. 10/050,879, filed Jan. 18, 2002. U.S. application Ser. No. 10/050,879 is a continuation-in-part of U.S. application Ser. No. 09/930,287, filed Aug. 16, 2001 and now abandoned, and claims the benefit under 35 USC 119(e) to provisional application No. 60/262,423, filed Jan. 19, 2001. U.S. application Ser. No. 09/930,287 claims the benefit under 35 USC 119(e) to provisional application No. 60/225,659, filed Aug. 16, 2000. Each of the above-identified applications is incorporated fully herein by reference.

### FIELD OF THE INVENTION

The present invention relates to a method and device for reversing and treating presbyopia.

### BACKGROUND OF THE INVENTION

Presbyopia affects virtually every person over the age of 44. According to Jobson Optical Database, 93% of people 45 and over are presbyopic. Presbyopia entails the progressive loss of amplitude of accommodation that occurs with aging. Adler's Physiology of the Eye, which is incorporated herein by reference, discloses that the human accommodative amplitude declines with age such that accommodation is substantially eliminated by the age of 50 to 55. Accommodative ability, as defined by U.S. Pat. No. 5,459,133 to Neufeld and incorporated in its entirety herein by reference for background information, is the capacity of the eye to focus for near vision by changing the shape of the lens to become more convex.

The ocular tissues involved in the accommodative response include the lens, the zonules, the lens capsule, and the ciliary muscle. Of these, the lens is the central tissue. These structures function together to enable the eye to focus on close objects by changing the shape of the lens. The lens is centrally suspended between the anterior and posterior chambers behind the pupillary opening of the iris. The lens is supported by an array of radially oriented zonular fibers, which extend from the lateral edges of the lens to the inner border of the circumferential ciliary muscle. The ciliary muscle is attached to the scleral coat of the eye. When the eye is at rest, it is focused for distance and the lens is in a somewhat flattened or less convex position. This shape is due to the tension that is exerted on the lens periphery by the zonules. The zonules pull the edges of the lens toward the ciliary body.

During accommodation, the shape of the lens becomes more convex through contraction of the ciliary muscle, which allows the ciliary attachment of the zonules to move toward the lens, reducing the tension in the anterior zonules. This reduction in tension allows the central region of the lens to increase in convexity, thereby enabling near objects to be imaged on the retina. The processes involving the coordinated effort of the lens, zonules, ciliary body, medial rectus muscles and iris, among others, that leads to the ability of the eyes to clearly focus near on the retina is the accommodative process.

Several theories have been advanced to explain the loss of accommodation with age. These theories include the hardening of the lens with age, loss of strength in the ciliary muscle, factors related to the physical growth of the lens, and, the loss of elasticity of the lens capsule. As for the loss of strength of the ciliary muscle, it is noted that although there are age-related morphological changes that occur, there is little evidence of diminishing strength of the ciliary muscle. In fact, under the influence of pilocarpine, the ciliary muscle will vigorously contract even in presbyopic eyes.

The lens grows throughout one's life and theories have been proposed that it is this increase in size that prohibits the effects of the zonules from affecting a change in the shape of the lens. Recent works exploring this possibility have not met widespread acceptance thus far. Most of the growth of the lens is not in its diameter, but instead, in its anterior-posterior dimensions.

As for changes in the lens capsule, it has been postulated that reduction in the elasticity of the capsule is, in fact, a contributing factor in presbyopia. Moreover, it has been found that Young's modulus of elasticity for the lens capsule decreases by nearly 50% from youth to age 60, while accommodation decreases by 98%. Consequently, the principal cause of presbyopia is now considered to be “lenticular sclerosis” or the hardening of the lens.

A cataract is a condition in which the lens becomes less clear. The study of cataracts lends insight into lens and capsular changes. The usual senile cataract is relatively discus-shaped when removed from the eye, its shape being dictated by the firm lens substance. The liquefied hypermature cataract is globular when extracted, rounded up by the elastic lens capsule. This is indirect evidence that it may be possible to reverse the lenticular changes associated with presbyopia, and that the lens capsule is still sufficiently elastic.

At the present time, common treatments for presbyopia include reading glasses, bifocal glasses, or mono-vision contact lenses. All of these solutions necessitate the use of an appliance creating additional shortcomings.

Alternative theories for treating presbyopia include scleral expansion and corneal reshaping. The efficacy of such techniques is not well-established and, importantly, these techniques do not attempt to reverse what the inventors of the subject-application believe to be a substantial causation, as explained more fully below, in the loss of the accommodative amplitude of the lens typically associated with the normal aging process. Moreover, because scleral expansion and corneal reshaping involve macroscopic changes in the morphology of the lens and/or cornea it fails to reverse presbyopia.

Finally, the use of the excimer laser for the purposes of corneal reshaping to produce a multifocal refracting surface has been disclosed in U.S. Pat. No. 5,395,356. While this method seems promising, it still requires structural changes to the cornea to compensate for aging changes in the lens. Rather than trying to undo the changes brought on by presbyopia, techniques such as these merely compensate for the loss of accommodative function by altering another ocular structure.

### SUMMARY OF THE INVENTION

While not wishing to be bound to any particular theory, it is now believed that presbyopia is caused by the hardening of the lens, which can be due to an alteration of the structural proteins or an increased adhesion between the lens fibers. It is also believed that the intralenticular viscosity increases with age as a result of the formation of certain chemical bond structures within the lens. Accordingly, the present invention is directed to method and apparatus for preventing and or reversing presbyopia through treatment of the lens such that the viscosity of the lens is reduced, restoring the elasticity and movement to the lens fibers and increasing the accommodative amplitude of the lens.

The claimed invention is also directed to a method of reversing or treating presbyopia resulting in underlying changes in the structures and/or interactions of molecules comprising those components of the eye associated with the accommodative process, most notably the lens and/or lens capsule.

In an embodiment, the present invention provides a novel molecular approach to reversing presbyopia by restoring the accommodative amplitude of the lens, and in another preferred embodiment, to reversing presbyopia while also reducing the tendency for the lens to lose its thus restored accommodative amplitude.

In another embodiment of the invention the onset of presbyopia is prevented by regularly administered treatment where elasticity and the accommodative ability of the lens is restored. By applying the treatments as described herein to the eyes of persons in their mid to late 30's, or even younger, the on-set of presbyopia, as defined by a loss of accommodation, such that the accommodative power of the eye is below 2.5 Diopters, can be avoided. In one embodiment of the invention, such treatments whether for the purposes of preventing or reversing presbyopia, would be occasionally repeated during the course of a patient's lifetime. The frequency of the treatment would be determined by the degree of accommodative loss that needs to be recovered, the amount of accommodation that can be safely restored in a single procedure, and the amount of restoration desired.

In one embodiment, the present invention is directed to a method for reversing and/or treating presbyopia by breaking disulfide bonds in molecules comprising the structures of the eye, most notably the lens and the lens capsule, in which disulfide bonds are believed to be a substantial factor in the progressive loss of accommodative amplitude. In another embodiment, the breaking of the disulfide bonds is accompanied by chemical modification of the sulfur moiety in the cysteine molecule formed upon breaking of the disulfide bonds, such chemical modification rendering the sulfur moiety less likely to form new disulfide bonds. This method thus comprises a method for preventing, and/or reducing the recurrence of presbyopia by reducing the probability of forming new disulfide bonds. Particularly, this invention affects a change in the accommodative amplitude of the human lens by: (1) using various reducing agents that cause a change in the accommodative abilities of the human lens, and/or (2) the use of applied energy to affect a change in the accommodative abilities of the human lens. It is believed that by breaking bonds, such as disulfides, that crosslink lens fibers together and increase lens viscosity causing a hardening of the lens cortex and lens nucleus, the present invention increases the elasticity and the distensibility of the lens cortex, lens nucleus, and/or the lens capsule.

Presbyopia, or the loss of the accommodative amplitude of the lens, has often advanced in a typical person age 45 or older to the point where some type of corrective lens in the form of reading glasses or other treatment is required. It is to be understood that loss of accommodative amplitude can occur in persons much younger or older than the age of 45, thus the present invention is not to be construed as limited to the treatment of presbyopia in a person of any particular age. The present invention is most useful in a person whose accommodative amplitude has lessened to a point where restoration thereof to some degree is desirable. However the invention should not be limited to the correction of presbyopia, but may be used to prevent presbyopia from occurring.

In one embodiment of the present invention, the method of reversing or preventing presbyopia will result in an increase in the accommodative amplitude at least about by 0.5 diopters. In another embodiment of the present invention, the method of reversing or preventing presbyopia will result in an increase in the accommodative amplitude of at least about 2.0 diopters. In still another embodiment, the method of reversing or preventing presbyopia of the present invention will result in an increase in the accommodative amplitude by at least about 5 diopters. In another embodiment of the present invention, the method of reversing or preventing presbyopia of the present invention will result in an increase of the accommodative amplitude of the lens to restoration thereof to that of a lens with a normal accommodative amplitude of 2.5 diopters or greater. It is noted that while it is obviously most beneficial to restore the accommodative amplitude of the lens to a normal accommodative amplitude, lesser degrees of restoration are also beneficial. For example, in some cases advanced presbyopia can cause severe reduction in the accommodative amplitude, thus making a complete restoration of the amplitude improbable.

### DETAILED DESCRIPTION

The accommodative amplitude of the lens is measured in diopters (D). The loss of accommodative ability begins at a very early age, such that by age 10 the average eye has 10 D, age 30, 5D, and by age 40, only 2.5D of accommodative power. The lens of a person who does not suffer from presbyopia (i.e. a person whose lens accommodates normally), will typically have an accommodative amplitude of about 2.5 diopters or greater. The terms “reversing presbyopia” or “treating presbyopia” as used herein mean increasing the accommodative amplitude of the lens.

As stated, inelasticity of the lens, or hardening thereof, is believed to be a contributing cause of presbyopia. The hardening of the lens can be due to an alteration of the structural proteins or an increased adhesion between the lens fibers. Additionally, it is believed that the lens viscosity also increases with age due to an increased concentration of certain chemical bond structures within the lens. In one embodiment, the present invention is directed to treating presbyopia by altering the molecular and/or cellular bonds between the cortical lens fibers so as to free their movement with respect to each other. The increased elasticity of the lens apparatus can restore lost amplitude of accommodation. Specifically, it is believed that disulfide bonds in the molecules comprising the structures of the eye responsible for proper accommodation are a substantial factor in the hardening of the lens and the concomitant loss of accommodative amplitude.

Thus, in one embodiment of the invention treatment process involves breaking the disulfide bond and then protonating the newly formed sulfur moiety with a reducing agent such as glutathione to impart a hydrogen atom thereto. The steps can be performed simultaneously or consecutively. In either case, the reducing agent can be present at the time the disulfide bond is broken in order to eliminate reformation of disulfide. That is, the reducing agent can introduce and bond a moiety onto the free sulfur after breaking the disulfide bond such that the likelihood of reformation of another disulfide bond is prevented or at least reduced. While the reducing agent may introduce a hydrogen atom onto the free sulfur, thus forming a sulfhydryl group (—SH), the resultant —SH groups can again be oxidized to form a new disulfide bond. Thus, it is advantageous to introduce a group into the free sulfur moiety, such as lower alkyls, methylating compounds, or other agents, which reduce the tendency of new disulfide bond formation. This method can result in a substantial prevention of the reoccurrence of presbyopia.

As stated, it is believed that the disulfide bonds form both between the lens fibers, between lens proteins, and between lens proteins and various thiols both within and on lens fibers. These bonds and substantially reduce the lens fibers' ability to easily move relative to each other and thus the ability of the lens to accommodate properly. While not wishing to be bound by any particular theory, the bonds may form by way of absorption of light energy, which causes the sulfhydryl bonds on the lens proteins to oxygenate removing a hydrogen atom from two adjacent —SH groups and creating water and a disulfide bond. Reducing the disulfide bonds requires hydrogen donors such as glutathione or other molecules. Other possible theories involve protein-thiol mixed disulfide bonds forming such as protein-S-S-glutathione or protein-S-S-cysteine. Glutathione therefore may be both part of the solution and part of the problem. The use of Glutathione in any treatment regimen therefore must be monitored carefully in light of the potential for an increase in undesirable bond formation.

The total refractive power of the lens is greater than what would be expected based on the curvature and the index of refraction. As stated, contraction of the ciliary muscle causes the ciliary body to move forward and towards the equator of the lens. This causes the zonules to relax their tension on the lens capsule, which allows the central lens to assume a more spherical shape. During accommodation, the main change is in the more central radius of curvature of the anterior lens surface, which is 12 mm in the unaccommodative state and can be 3 mm centrally during accommodation. Both the peripheral anterior and the posterior lens surfaces change very little in curvature during accommodation. The axial thickness increases while the diameter decreases. The central anterior lens capsule is thinner than the rest of the anterior capsule. This may explain why the lens bulges more centrally during accommodation. The thinnest portion of the capsule is the posterior capsule, which has a curvature greater than the anterior capsule in the unaccommodative state. The protein content of the lens, almost 33% by weight, is higher than any other organ in the body. There are many chemical compounds of special interest in the lens. For example, glutathione is found in high concentration in the lens cortex even though there is very little in the aqueous. Thus, the lens has a great affinity for glutathione and actively absorbs, transports and synthesizes glutathione. Approximately 93% of intralenticular glutathione is in the reduced form. Glutathione may be involved with maintaining the lens proteins, the sulfhydryl groups (—SH), in their reduced states. That is, after the disulfide bond is broken and the sulfur moieties are made available, glutathione can impart a hydrogen atom to form the sulfhydryl group thereby preventing or minimizing the reformation of a disulfide bond. In addition, ascorbic acid can also be found in very high concentrations in the lens. It is actively transported out of the aqueous and is at concentrations 15 times that found in the bloodstream. Both inositol and taurine are found at high concentrations in the lens for which the reason is not known.

According to one embodiment of the invention, the increase in the accommodative amplitude is accomplished by treatment of the outer lens region (the cortex) or the inner layer (the nucleus) with radiation, sonic or electromagnetic energy, heat, chemical, particle beam, plasma beam, enzyme, gene therapy, nutrients, other applied energy source, and/or any combination of any of the above sufficient to break the disulfide bonds believed responsible for the inelasticity of the lens. Chemicals are useful to reduce disulfide bonds that are believed to anchor lens fibers hence preventing their free movement and elasticity. By making the anterior cortex and/or the nucleus more elastic, viscosity is lowered and the lens is again able to assume its characteristic central bulge during accommodation.

Chemicals suitable for causing reduction include, by way of example only, glutathione, ascorbic acid, Vitamin E, tetraethylthiuram disulfyl, i.e., reducing agent, any biologically suitable easily oxidized compound, ophthalmic acid, inositol, beta-carbolines, any biologically suitable reducing compound, reducing thiol derivatives with the structure: 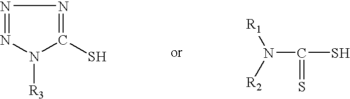
or sulfur derivatives with the structures: 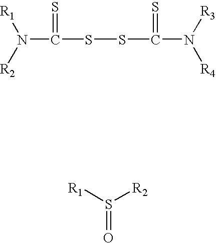
wherein R₁, R₂, R₃ and R₄ are independently a straight or branched lower alkyl that may be substituted, e.g., by hydroxyl, lower alkoxy or lower alkyl carbonyloxy, their derivatives or a pharmaceutically acceptable salt thereof. Preferred exemplary reducing agents include diethyl dithiocarbamate, 1-methyl-1H-tetrazol-5-yl-thiol and 1-(2-hydroxyethyl)-1H-tetrazol-5-yl-thiol or and pharmaceutically acceptable salts thereof. Other useful compounds can be found in U.S. Pat. No. 5,874,455, which is hereby incorporated in its entirety by reference for background information. The above-mentioned chemicals are merely exemplary and other reducing agents that behave similarly by breaking the disulfide bond are included within the scope of this invention.

The chemical reducing agents can be used alone or in conjunction with a catalyst such as an enzyme. Enzymes and other nutrients suitable for causing or facilitating reduction include, for example, aldoreductase, glyoxylase, glutathione S-transferase, hexokinase, thiol reductase, thioltransferase, tyrosine reductase or any compatible reductase. The need for a source of applied energy for the reduction of the disulfide bonds may be met by the addition of glucose-6-phosphate, which is present within the lens but the enzyme, hexokinase that normally converts the glucose to the G6P energy state is rendered non-functional by the process of thiol oxidation. Again, it should be noted that the above-listed enzymes are exemplary and not an exhaustive list. The enzymes can be naturally present in the eye, or can be added to the eye together with or separate from the chemical reducing agent or energetic means disclosed herein. As such, other chemically and biologically comparable enzymes that help break disulfide bonds or behave similarly should be considered as within the scope of the present invention.

In one embodiment of the invention, the reduction of disulfide groups of the lens proteins to sulfhydryl groups is accomplished by delivering to the lens a compound such as glutathione, thiols, or others in sufficient quantities to reduce the disulfide bonds and other molecular and cellular adhesions. Other enzymes or chemicals that affect a methylation on the free sulfur atom include for example, methyl-methane thiosulfonate, methyl glutathione, S-methyl glutathione, S-transferase and other biologically compatible methylating agent. Use of emulsions such as nanocapsules, albumin microspheres, carrier molecules such as inositol, taurine or other biologically suitable means such as virus phages for delivering the reducing agent or enzymes to the lens is an integral part of this invention. The chemical reducing agent will typically be delivered in the form of a solution or suspension in an ophthalmically acceptable carrier. In some cases, the application of energy to affect or catalyze the reduction of the disulfide bonds as well as the disruption of other bonds and adhesions may be beneficial. The application of energy alone can be used to break the disulfide bonds. Applied energy can have any form, by way of example only, any of laser, ultrasound, particle beam, plasma beam, X-ray, ultraviolet, visible light, infrared, heat, ionizing, light, magnetic, microwave, sound, electrical, or other not specifically mentioned, can be used alone or in combination with the reducing agents to affect the treatment of presbyopia, or a combination of any of these types of energies.

In a similar manner, agents can be delivered to the lens capsule, which bind or interact with the capsule to affect greater elasticity or distensibility. Such agents either cause the capsule to shrink in surface area or increase the tension of the lens capsule on the peripheral anterior or posterior of the lens. Applied energy can have any form, by way of example only, any of laser, ultrasound, heat, particle beam, plasma beam, X-ray, ultraviolet, visible light, infrared, ionizing, light, magnetic, microwave, sound, electrical, or other not specifically mentioned can be used alone or in combination with the reducing agents to affect the treatment of presbyopia or a combination of any of these types of applied energy.

In another embodiment of the invention, applied energy can be used as a catalyst to induce or increase the rate of the reduction reaction. Thus, by applying energy, the peripheral portion of the capsule is preferentially affected, leaving the central 4 mm zone of accommodation unaffected. This allows the lens to assume a more accommodative state. The applied energy can also be applied alone to promote the reduction reaction and the cellular changes that ultimately affect the lens' cortex. As examples, lasers useful in the present invention include: excimer, argon ion, krypton ion, carbon dioxide, helium-neon, helium-cadmium, xenon, nitrous oxide, iodine, holmium, yttrium lithium, dye, chemical, neodymium, erbium, ruby, titanium-sapphire, diode, femtosecond or attosecond laser, any harmonically oscillating laser, or any other electromagnetic radiation. Exemplary forms of heating radiation include: infrared, heating, infrared laser, radiotherapy, or any other methods of heating the lens. Finally, exemplary forms of sound energy that can be used in an embodiment of the invention include: ultrasound, any audible and non-audible sound treatment, and any other biologically compatible sound energy.

In still another embodiment of the present invention, radiation, such as ultraviolet light, visible light, infrared, microwave, or other electromagnetic energy may be placed in the eye to help break the disulfide bonds. This would then make it possible for the reduction of the disulfide bonds to occur.

The applied energy used with various embodiments and methods of the present invention could be applied through either contact with the sclera or cornea, non-contact techniques, or through intraocular methods of delivery. More than one treatment may be needed to affect a suitable increase in the accommodative amplitude. When more than one modality of treatment is desirable, chemical treatment can be administered prior to, after, or simultaneously with the application of energy.

Embodiments of the present invention further relate to a pharmaceutical agent capable of crossing an outer surface of an eye to affect an accommodative ability of the eye by decreasing aberrant lenticular bonds in the eye. More specifically, the pharmaceutical agent may be capable of penetrating the cornea and affecting the eye to increase its accommodative ability, both with and without the addition of an energy source. Still more specifically, the pharmaceutical agent may affect the anterior lens surface of the eye to increase accommodation. The pharmaceutical agent may act to decrease or eliminate the aberrant biochemical bonds responsible for the loss of elasticity in the lens. As discussed earlier, such aberrant biochemical bonds may cause adhesion between lens fibers, leading to reduced elasticity and accommodative ability of the lens. The aberrant biochemical bonds may include or be formed by, as an example only, covalent attachments of a variety of sugar resides to form glycoproteins, the addition of phosphate groups (PO₄²⁻) or sulfate groups (SO₄²⁻) to tyrosine (one of the amino acids that make up most proteins), or disulfide bonds between neighboring cysteine amino acids. The aberrant biochemical bonds may include any kind of oxidized bond, of which disulfide bonds are only one example.

In embodiments, the pharmaceutical agent may be a pro-drug. The meaning of “pro-drug” as used here includes having the property of being changeable from an inactive state to an active state. In its inactive state, a pro-drug may be able to cross a membrane of the body more easily than in its active state, making the inactive pro-drug more easy to deliver to a specific site. Once at the site, however, the pro-drug may be caused to assume an active state that allows the pro-drug to generate whatever therapeutic effect it is intended for. Activating the pro-drug—i.e., causing the pro-drug to assume the active state—may involve altering the chemical properties of the compound or compounds constituting or present in the pro-drug. Thus, the meaning of “pro-drug” further encompasses having the capability of being converted or transformed from a first biochemical or pharmacological substance to a second biochemical or pharmacological substance with properties different from properties of the first substance.

In the case of the eye, a reducing substance may be applied in pro-drug form to the outer eye, for example in a drop of liquid. The pro-drug may be in an inactive state when initially applied. The inactive state of the pro-drug may enable the pro-drug to more easily cross from the outer surface of the eye into the inner eye than would be the case it the pro-drug were in an active state. More specifically, the pro-drug may cross from the outside of the cornea to inside the aqueous humor of the eye. The pro-drug may further have solubility or acid/base properties, for example, that enable it cross the corneal boundary. An example of a pro-drug agent is N-acetylcarnosine. Substances such as N-acetylcarnosine have the ability to cross the cornea and then be converted into other agents, such as carnosine, in the anterior chamber

Once inside the eye, the pro-drug may be activated/converted. In its active state/converted form, the pro-drug may act as a reducing agent. To this end, the pro-drug in its active state may comprise reducing compounds. The transition of the pro-drug from its inactive state to its active state may be caused by one or more of a number of factors. For example, naturally occurring enzymes in the aqueous humor of the eye could cause the transition. Alternatively or additionally, energy could be applied externally as described earlier. That is, radiation, sonic or electromagnetic energy, heat, chemical, particle beam, plasma beam, enzyme, gene therapy, nutrients, other applied energy source, and/or any combination of any of the preceding could be applied. The applied energy may both cause a transition of the pro-drug from an inactive state to an active state, and break the aberrant biochemical bonds believed responsible for the inelasticity of the lens. The active pro-drug may then work to reduce the broken bonds. The active pro-drug may in particular be a substance for which the lens has an affinity, so that the lens actively takes up the pro-drug once past the cornea and inside the eye.

According to further embodiments of the present invention, an enzyme or enzymes may also be introduced into an eye in pro-drug form. For example, a large enzyme, such as thiolreductase, could by applied in an eye drop. In the eye drop, the large enzyme may be in a disassembled form, rendering it inactive. The disassembled form of the enzyme may make it easier for the enzyme to cross the corneal boundary into the inner eye and be taken up by the lens. Once in the inner eye, the enzyme could be activated. Here, activation may involve the re-assembly of the enzyme constituents. Activation may be brought about through the use of various externally applied forms of energy, as discussed above, or by way of the various intralenticular enzymes already present within the lens. Once assembled within the lens, the enzyme may act to promote a reduction reaction by breaking aberrant biochemical bonds including but not limited to disulfide bonds, and transferring a reducing molecule (proton) from a reducing compound to the broken bonds to prevent reformation of the bonds. Accordingly, a reducing agent to supply the reducing molecule may be introduced concurrently with the enzyme. Alternatively, the reducing agent may be introduced before or after the introduction of the enzyme. The reducing agent may be in pro-drug form.

According to alternative embodiments of the present invention, a pharmaceutical agent capable of affecting the eye's accommodative ability may be introduced by direct injection. The pharmaceutical agent may include an enzyme or enzymes to promote a reduction reaction, and/or a reducing substance. The injection may be, for example, directly into the lens or into the anterior chamber, into the vitreous or the posterior chamber. The approach for this injection may be, for example, through the cornea or through the sclera. The injection may be followed by the external application of energy to further promote a reduction reaction.

If not injected into the lens but instead, for example, into the aqueous humor, the injected substance may be capable of crossing a lenticular capsular boundary into the lens. For example, the injected substance may be a substance for which the lens has an affinity, so that the lens actively takes up the substance once inside the eye. The injected substance may further be capable, upon entering the lens, of being converted into a second substance capable of affecting an accommodative ability of the eye, such as a reducing substance.

Since most of the naturally occurring lens shape alteration occurs in the central anterior portion of the lens when the eye refocuses from distant objects to near objects, lessening of aberrant lenticular bonds may be particularly beneficial when it occurs at the anterior central lens, and more specifically at the region of the anterior central lens which changes its topography during accommodation. The anterior central region of the lens is also the most easily reached by both drugs and by application of energy. Thus, embodiments of the present invention relate to specifically targeting the anterior central region of the lens for treatment. On the other hand, embodiments of the present invention further relate to specifically targeting regions outside of the anterior central region of the lens for treatment. Still further, embodiments of the present invention relate to specifically targeting the anterior central region of the lens as well as regions outside of the anterior central region of the lens for treatment.

Targeting applications according to embodiments of the present invention may include, for example, applying a reducing substance to an eye in a non-selective or non-targeted fashion, for example with an eye drop. The reducing substance may be capable of crossing the corneal boundary into the inner eye. Then, energy may be applied to only a selected portion of the lens, such as the anterior central region of the lens. In such targeting applications, the reducing agent could be formulated so that it was inactive to reduce aberrant lenticular bonds in the absence of the application of energy, but so that, when energy was applied, the reducing agent became activated and able to reduce broken lenticular bonds. Thus, the targeted or focused application of energy would break lenticular bonds in a selected portion of the lens, and also activate reducing agent present in the selected portion. A reducing agent with the latter properties could be obtained by one skilled in the art, for example, by suitably selecting the agent, by suitably formulating constituent compounds of the agent, by suitably controlling the concentration of the agent or of respective constituent compounds thereof, or by any combination of the foregoing. The reducing agent could be, for example, a pro-drug. On the other hand, the reducing agent could be in active form as applied, be capable of crossing the corneal boundary into the inner eye, and not require the application of energy to become active.

Targeted treatment as described above addresses an important consideration involved in the treatment of presbyopia by decreasing or lessening aberrant lenticular bonds, in that there are many important and necessary bonds in the lens that should not be altered. For example, the disulfide bond is one of the fundamental protein bonds responsible for much of the three-dimensionality of enzymes and proteins. If all the disulfide bonds were eliminated (e.g., by breaking and reducing the bonds), then there would be no real three-dimensional architecture to the lens. The lens might at this point be more of a capsular bag of fluid. On the other hand, the disulfide bond, as discussed earlier, is believed to be a factor in causing presbyopia. Using targeted treatment as described above, the amount of decrease or lessening of lenticular bonds may be controlled to be within specific ranges, within specific portions of the eye, thereby preserving beneficial bonds while reducing others selectively. In one range, aberrant lenticular bonds may be decreased by 10% to 70% in targeted regions. In another range, aberrant lenticular bonds may be decreased by 20%-50% in targeted regions.

In embodiments of the present invention, targeted treatment need not include application of a reducing agent in conjunction with the application of energy. Instead, a substance could be suitably formulated (e.g., in terms of constituent compounds, concentration, etc. as described above) so that the substance could both break aberrant biochemical bonds and reduce the broken bonds, within specific portions of the eye. For example, the specific portion could be in the anterior central region of the eye. To target a specific portion of the eye, the substance could, for example, be formulated to have an affinity for the specific portion. The amount of decrease in lenticular bonds could be controlled to be within specific ranges. In one range, aberrant lenticular bonds may be decreased by 10% to 70% in targeted regions. In another range, aberrant lenticular bonds may be decreased by 20%-50% in targeted regions.

In embodiments of the present invention, iontophoresis may be used to help transport a reducing substance into the eye and into the lens. Application of energy may or may not be used in conjunction with the latter.

In still other embodiments of the present invention, one or more enzymes to promote or facilitate a reduction reaction may be introduced into the lens using a viral phage. The viral phage may be used to transfect the lens cells with a gene to transcribe an enzyme's genetic code into the lens cells using the RNA or DNA transcriptase already present within the lens cells, as opposed to introducing the enzyme itself. Once this genetic code was in the lens cells, the natural protein manufacturing mechanisms present in the cells would create the enzyme from the genetic code. This technique would circumvent the issue of getting large enzymes into the lens through the lens capsule. The lens could then further be treated with reducing agents in any form, and applications of energy including energy targeted or focused on specific portions of the lens.

Finally, it is observed that various types of human tissues are derived from the same epithelial line of embryonic cells as the lens. The skin is one example. The skin undergoes various forms of oxidation, which leads to the typical alterations brought on by aging. Methods as described above could be applied to epithelial tissues like skin to reduce the oxidized biochemical bonds therein, such as the disulfide bonds, to thereby rejuvenate the tissues. Each of the various epithelial tissues could receive a treatment specifically designed to take into account the tissue's location and accessibility. For example, the skin can be treated directly with a reducing agent and then energy could be applied to help break the oxidized bonds. Additionally or alternatively, a combination of enzymes and catalysts can be used to stimulate the reduction reaction. Specific portions of the skin or other epithelial tissue could be treated with targeted application of energy.

The reducing agents and enzymes used to treat the skin or other epithelial tissue as described above could be applied in active form, or could have any one or any combination of the properties of the reducing agents and enzymes discussed above in connection with treatment of the eye for presbyopia. That is, the reducing agents and enzymes could be in pro-drug form, or in a form requiring the application of energy to become active, or the like.

Several embodiments of the present invention are specifically illustrated and described herein. However, it will be appreciated that modifications and variations of the present invention are covered by the above teachings and within the purview of the appended claims without departing from the spirit and intended scope of the invention.

1. A method comprising applying a pharmaceutical agent to an outer surface of an eye, the pharmaceutical agent capable of crossing the outer surface to affect an accommodative ability of the eye by decreasing aberrant lenticular bonds in the eye.

2. The method of claim 1, wherein the aberrant lenticular bonds are oxidized bonds.

3. The method of claim 1, wherein the pharmaceutical agent has a first form to facilitate crossing the outer surface, the first form being convertible after crossing the outer surface into a second form, the second form including a biochemical substance capable of affecting the accommodative ability of the eye.

4. The method of claim 3, further comprising applying energy to the eye to convert the pharmaceutical agent from the first form into the second form.

5. The method of claim 3, wherein the pharmaceutical agent is capable of being converted into the second form by naturally occurring enzymes in the aqueous humor of the eye.

6. The method of claim 3, wherein the biochemical substance includes a reducing substance.

7. The method of claim 3, wherein the biochemical substance includes an enzyme that facilitates a reduction reaction.

8. The method of claim 7, wherein the first form comprises the enzyme in disassembled form, and the enzyme is re-assembled into an active enzyme within the eye.

9. The method of claim 1, wherein the pharmaceutical agent is a pro-drug.

10. A method comprising:
applying a pharmaceutical agent to an eye, the pharmaceutical agent being adapted to affect an accommodative ability of a lens of the eye; and
applying energy to a specific portion of the eye to cause the pharmaceutical agent to affect the accommodative ability.

11. The method of claim 10, wherein the pharmaceutical agent comprises a reducing substance, and the applied energy causes a reduction reaction to occur.

12. The method of claim 11, wherein the applied energy breaks oxidized lenticular bonds and the reducing substance reduces the broken oxidized lenticular bonds.

13. The method of claim 10, wherein the specific portion is the anterior central region of the lens.

14. The method of claim 10, wherein the specific portion is outside the anterior central region of the lens.

15. The method of claim 10, wherein in the specific portion of the eye, aberrant lenticular bonds are decreased by 10% to 70%.

16. The method of claim 10, wherein in the specific portion of the eye, aberrant lenticular bonds are decreased by 20% to 50%.

17. The method of claim 11, wherein the reducing substance is inactive in the absence of the application of energy.

18. The method of claim 11, wherein the reducing substance includes glutathione.

19. The method of claim 11, wherein the reducing substance includes N-acetylcarnosine.

20. A method for treating presbyopia, comprising injecting a first biochemical substance into the aqueous humor of an eye, the first biochemical substance capable of crossing a lenticular capsular boundary, the first biochemical substance further being capable of being converted into a second biochemical substance capable of affecting an accommodative ability of the eye

21. The method of claim 20, wherein the second biochemical substance includes a reducing substance.

22. The method of claim 20, wherein the second biochemical substance includes an enzyme to facilitate a reduction reaction.

23. The method of claim 20, wherein the first biochemical substance includes an viral phage containing genetic information to generate an enzyme that facilitates a reduction reaction.

24. The method of claim 20, wherein the first biochemical substance includes a glutathione derivative and the second biochemical substance includes a reduced glutathione.

25. The method of claim 20, wherein the first biochemical substance includes N-acetylcarnosine and the second biochemical substance includes carnosine.

26. A method to bring about the reduction of oxidized epithelial tissues throughout the body by applying a biochemical substance to the skin or epithelial tissue as either a pro-drug or as an active drug, the biochemical substance being adapted to affect an reduction of oxidized bonds; and applying energy to a specific portion of the skin or epithelial tissue to cause a reduction reaction by the reducing substance to affect the reduction of the oxidized bonds.

27. The method of claim 26, wherein the bonds to be reduced are disulfide bonds.

28. The method of claim 26, wherein the energy is any form of electromagnetic energy.

29. The method of claim 26, wherein the electromagnetic energy is one or more of laser energy, visible light energy, ultraviolet light energy, infrared energy, of microwave energy.

30. The method of claim 26, wherein the biochemical substance includes a reducing compound.

31. The method of claim 26, wherein the biochemical substance includes an enzyme.

32. The method of claim 26, wherein the biochemical substance is activated by the applied energy.

33. The method of claim 26, wherein the energy breaks the oxidized bonds.

34. A method for treating presbyopia, comprising:
applying a biochemical substance to an eye, the substance being capable of crossing an outer surface of the eye to enter an inner part of the eye; and
causing a transition in a state of the substance from an inactive state to an active state wherein the substance is capable of affecting an accommodative ability of the eye.

35. The method of claim 34, wherein to affect the accommodative ability of the eye the substance is capable of reducing aberrant lenticular bonds in the eye.

36. The method of claim 34, wherein to affect the accommodative ability of the eye the substance is capable of promoting a reduction reaction in the eye.

37. The method of claim 33, where substance includes an enzyme.

38. The method of claim 34, wherein the transition is caused by naturally occurring enzymes in the eye.

39. The method of claim 31, wherein the transition is caused by the application of external energy.

40. The method of claim 31, wherein the biochemical substance includes N-acetylcarnosine.

41. A method for treating presbyopia, comprising:
applying a reducing agent to an eye; and
focusing energy on a specific portion of the eye to break lenticular bonds in the specific portion.

42. The method of claim 41, wherein the reducing agent is active to reduce broken lenticular bonds without the focused energy.

43. The method of claim 41, wherein the focused energy activates the reducing agent to reduce broken lenticular bonds.

44. The method of claim 41, wherein the reducing agent includes glutathione.

45. The method of claim 44, wherein the energy is an ultraviolet laser.

46. The method of claim 44, wherein the energy is a visible light laser.

47. The method of claim 41, wherein the reducing substance includes N-acetylcarnosine.

48. The method of claim 47, wherein the energy is an ultraviolet laser.

49. The method of claim 47, wherein the energy is a visible light laser.

50. A method for treating presbyopia, comprising applying a substance to the eye, wherein the substance is formulated to affect an accommodative ability of the eye within a specific portion of the eye.

51. The method of claim 50, wherein the substance is capable of breaking and reducing aberrant biochemical bonds including but not limited to disulfide bonds within the specific portion.

52. The method of claim 50, wherein the substance has an affinity for the specific portion.

53. The method of claim 50, wherein the specific portion is in the anterior central region of the eye.

54. The method of claim 50, wherein in the specific portion of the eye, 10% to 70% of aberrant biochemical bonds including but not limited to disulfide bonds are broken and reduced.

55. The method of claim 50, wherein in the specific portion of the eye, 20% to 40% of aberrant biochemical bonds including but not limited to disulfide bonds are broken and reduced.

56. A method comprising injecting a pro-drug agent containing a biochemical substance capable of affecting an accommodative ability of a lens of the eye into an eye.

57. The method of claim 56, wherein the biochemical substance reduces aberrant lenticular bonds in the lens.

58. The method of claim 56, further comprising applying energy to the eye to activate a reduction reaction

59. A method comprising using iontophoresis to facilitate introducing a biochemical substance capable of affecting an accommodative ability of a lens of an eye across a corneal boundary.

60. The method of claim 58 wherein the biochemical substance reduces aberrant lenticular bonds.

61. A method comprising using a viral phage to facilitate introducing a biochemical substance capable of affecting an accommodative ability of a lens of an eye across a corneal boundary.

62. The method of claim 61, wherein the biochemical substance comprises the genetic code of an enzyme.

63. The method of claim 61, wherein the viral phage transfects cells of the lens with a gene to transcribe the genetic code into cells of the lens.

64. The method of claim 63, wherein the genetic code transcribes thioltransferase.

65. The method of claim 63, wherein the genetic code transcribes hexokinase.

66. The method of claim 63, wherein the genetic code transcribes glutathione reductase.

67. The method of claim 61, further comprising treating the eye with a reducing agent.

68. The method of claim 67, wherein the reducing agent is reduced glutathione.

69. The method of claim 67, wherein the reducing agent is reducing thiol derivatives.

70. The method of claim 67, wherein the reducing agent is substituted indoles.

71. The method of claim 67, further comprising applying energy to the eye.

72. The method of claim 71, wherein the energy is an ultraviolet laser.

73. The method of claim 71, wherein the energy is a visible light laser.

74. A method comprising applying a reducing agent to a human epithelial tissue, the reducing agent being adapted to reduce a biochemical bond in the tissue.

75. The method of claim 74, further comprising applying energy to the epithelial tissue to activate the reducing agent.

76. A pharmacological composition for the treatment of presbyopia, comprising a pharmaceutical agent capable of crossing an outer surface of an eye to affect an accommodative ability of the eye by decreasing aberrant lenticular bonds in the eye.

77. The pharmacological composition of claim 76, wherein the pharmaceutical agent is convertible by the application of energy into a biochemical substance capable of affecting an accommodative ability of the eye.

78. The pharmacological composition of claim 77, wherein the pharmaceutical agent is capable of being converted into the biochemical substance by naturally occurring enzymes in the aqueous humor of the eye.

79. The pharmacological composition of claim 78, wherein the pharmaceutical agent includes N-acetylcarnosine.

80. The pharmacological composition of claim 78, wherein the pharmaceutical agent includes any glutathione derivative.

81. The pharmacological composition of claim 76, wherein the biochemical substance includes a reducing substance.

82. The pharmacological composition of claim 81, wherein the reducing substance includes reduced glutathione.

83. The pharmacological composition of claim 81, wherein the reducing substance includes reducing thiol derivatives.

84. The pharmacological composition of claim 81, wherein the reducing substance includes substituted indoles.

85. The pharmacological composition of claim 81, wherein the reducing substance includes reduced carnosine.

86. The pharmacological composition of claim 76, wherein the biochemical substance includes an enzyme that facilitates a reduction reaction.

87. The pharmacological composition of claim 86, wherein the enzyme includes thioltransferase.

88. The pharmacological composition of claim 86, wherein the enzyme includes hexokinase.

89. The pharmacological composition if claim 86, wherein the enzyme includes glutathione reductase.

90. The pharmacological composition of claim 86, wherein the enzyme includes glutathione-S-transferase.

91. The pharmacological composition of claim 86, wherein the pharmaceutical agent comprises the enzyme in disassembled form, and the enzyme is re-assembled into an active enzyme within the eye.

92. A pharmacological composition for the treatment of an epithelial tissue, comprising a biochemical substance adapted to effect a reduction of aberrant oxidized bonds in the tissue.

93. The pharmacological composition of claim 92, wherein biochemical substance is capable of being activated by the application of energy to cause a reduction reaction by the biochemical substance to effect the reduction of the oxidized bonds.

94. The pharmacological composition of claim 92, wherein the biochemical substance includes a reducing agent.

95. The pharmacological composition of claim 92, wherein the biochemical substance includes an enzyme.

96. The pharmacological composition of claim 92, wherein the biochemical substance includes a pro-drug.
